(19) United States
(12) Patent Application Publication
(10) Pub. No.: US 2024/0207512 A1
(43) Pub. Date: Jun. 27, 2024

(54) DEVICE AND METHOD FOR ADMINISTERING LIQUIDS, COMPRISING A DISPLAY UNIT FOR GRAPHICALLY REPRESENTING THE FILLING LEVEL

(71) Applicant: B. Braun Melsungen AG, Melsungen (DE)

(72) Inventors: Klaus Angersbach, Morschen (DE); Christoph Erlen, Kassel (DE)

(21) Appl. No.: 17/918,351

(22) PCT Filed: Feb. 22, 2022
(86) PCT No.: PCT/EP2022/054348

§ 371 Date: Oct. 12, 2022

(30) Foreign Application Priority Data
Feb. 24, 2021 (DE) 10 2021 104 411.1

(51) Int. Cl. A61M 5/172; A61M 5/145

(52) U.S. Cl. CPC A61M 5/172; A61M 5/1452; A61M 2205/3389; A61M 2205/505; A61M 2205/584

(57) ABSTRACT

A device and method for administering a medical liquid. The device includes a receiving region for receiving a hollow body containing the medical liquid and a cover connected to the receiving region. The cover is movable between an opened position and a closed position. The device also includes a drive configured for acting on the hollow body such that the medical liquid is conveyed. The device further includes a display unit for graphically displaying the filling level of the hollow body based on one or more operating parameter(s) of the drive, especially operating parameter(s) relevant to conveying the liquid.

### CROSS-REFERENCE TO RELATED APPLICATIONS

This application is the United States national stage entry of International Application No. PCT/EP2022/054348, filed Feb. 22, 2022, and claims priority to German Application No. 10 2021 104 411.1, filed Feb. 24, 2021. The contents of International Application No. PCT/EP2022/054348 and German Application No. 10 2021 104 411.1 are incorporated by reference herein in their entireties.

### FIELD

The present disclosure relates to a device for administering a medical liquid, in particular a syringe pump. Further, the present disclosure relates to a method (for operating a device) for administering a medical liquid.

### BACKGROUND

When devices for administering a medical liquid, in particular syringe pumps, are used, it is essential for the user to be aware at all times of how much liquid is left in the hollow body, in particular in the syringe. In particular, it must be possible to exclude that the fill volume of the syringe falls below a critical limit, as this might result in a wrong dosing of drugs and even in live-threatening situations.

In known syringe pumps, this can be ensured, for example, by an operating field of the syringe pump and a loaded syringe being freely accessible and visible at any time so that the filling level can be read directly on the syringe and, at the same time, the operating field is visible for displaying or controlling data relevant to conveying. It is, however, a drawback in this context that, due to its design in which the operating field and the syringe are superimposed or juxtaposed to be freely accessible, the syringe pump has no compact structure and occupies a relatively large proportion of the space that is already limited in an intensive care unit.

Also, there are known syringe pumps in which the syringe can be loaded only when the operating front is unfolded and during operation is located behind the operating front. This offers the advantage, to be sure, that a compact space-saving syringe pump can be provided, wherein the filling state of the syringe can only be viewed directly when the operating front is opened. The drawback is that the operating front for displaying or controlling data relevant to conveying cannot be observed or set simultaneously with viewing the filling level.

In addition, there are syringe pumps known from DE 20 209 581 U1, for example, in which the loaded syringe can only be loaded when the operating front of the syringe pump is unfolded, and in the closed state is located behind the operating front. The filling level of the syringe can be read through a window in the operating front. Alternatively, a region can be spared in the operating front so that the filling level and the operating front are visible simultaneously. This in turn has the drawback, however, that the syringe pump as such requires a larger design, or that, when the syringe pump has a compact design, the visibility of the syringe must be made possible at the expense of the size of the operating front. Since the operating front should be as large as possible, i.e., should particularly have a large display, for modern flexible and intuitive operation, providing a window or a recess in the operating front for directly viewing the syringe filling level should be avoided.

For example, from EP 3 266 481 A1 an infusion system is known in which a liquid-gas interface of a drip chamber is displayed on a display unit. The liquid filling level is determined by an optical sensor or by a mass or weight measurement along with a liquid injection rate, for example, and is graphically shown on the display unit. It is a drawback, however, that the filling level cannot be read directly on the liquid container so that the display may not be intuitive for a user accustomed to the known syringe pumps. Moreover, there is the problem that determining the filling level requires additional sensors and/or may be inaccurate or wrong due to the weight measurement and the optical measurement, e.g., when the liquid container is tilted.

Moreover, from WO 2014/100 736 A2 a syringe pump comprising a receiving region, a cover, a drive and a display unit is known.

### SUMMARY

Therefore, it is the object of the present disclosure to provide a compact device for administering a medical liquid with maximum possible functionality in which simple and intuitive operability as well as precise monitoring of the filling level of liquid is safeguarded at any time.

In particular, the device for administering a medical liquid is designed as a syringe pump. A syringe pump is understood to be a dosing pump by which the medical liquid contained in a syringe can be continuously administered to a patient by axial movement of a plunger/piston of the syringe. One example is a syringe pump available from B. Braun under the registered trademark PERFUSOR®.

The device has a receiving region which is suitable for receiving at least in portions a hollow body containing the liquid. That is to say that the medical liquid to be administered is filled into the hollow body and the hollow body is received in the receiving region of the device for administering the medical liquid. In the case of a syringe pump, the hollow body is designed as a cylindrical hollow body of a syringe.

The device has a cover which is movably, such as pivotably, connected to the receiving region between an opened position and a closed position. In the opened position of the cover, the receiving region is accessible and/or visible. That is, in the opened/unfolded position of the cover, the hollow body can be loaded into the receiving region, and/or a filling level of the liquid contained in the hollow body can be read (directly on the hollow body) in the opened/unfolded position of the cover. The cover can be used to retain the hollow body in the receiving region and/or to protect the hollow body from external influences. In the closed position of the cover, the receiving region, at least the filling level of the hollow body, is covered to the outside. That is to say that the filling level is covered in the closed/folded position of the cover so that it cannot be read from the outside by a user and/or cannot be removed from the device without moving or pivoting the cover, at least not without damage.

The device has a drive/an actuator, which is suitable for acting on the hollow body received in the receiving region such that the liquid contained in the hollow body is conveyed. In particular, the drive is suitable for axially moving a plunger of the syringe so that the contained liquid is pressed out of the syringe.

The device has a display unit for graphical representation of the filling level of the hollow body, for example in the form of a display unit. The display unit is suitable for displaying the filling level of the hollow body based on one or more operating parameter(s) of the drive particularly relevant to conveying the liquid.

As the one or more operating parameter(s) of the drive particularly relevant to conveying has/have a direct influence on the filling level of the hollow body, it/they can be used to precisely indicate the current filling level. It is another advantage that the operating parameter of the drive must be detected and monitored for correctly dosing and conveying the liquid to be administered anyway so that no additional sensors are required. By visualizing the filling level on the display unit, it is no longer required to read the filling level directly on the hollow body so that the hollow body can be disposed in an area of the device that is not visible/accessible in a space-saving manner without negatively affecting the functionality.

According to a preferred embodiment, the display unit can be disposed on an outside of the device, in particular integrated as an operating front on the cover/in the cover. Preferably, the display unit can extend, in the closed position of the cover, completely on an outside of the device, in particular on a front outside in the position of use of the device. As an alternative, the display unit can also be designed separately from the cover and, for example, on a central monitor, such as for monitoring plural devices. In addition, it is imaginable that the display unit can be coupled to an external display device so that the filling level can be additionally graphically displayed on the external display device which is not part of the device for administering the liquid.

According to a preferred embodiment, the display unit may be used to display information for the user and to input user commands and may be designed particularly in the form of a touch-controlled/touch-sensitive touch display/touch screen.

According to a preferred embodiment, the device may include a control unit for controlling the drive. Thus, the conveying of the liquid to be administered can be precisely set and controlled.

According to a preferred embodiment, the drive can be designed as a linear drive comprising a drive motor and a spindle coupled to the drive motor and can be a translation position of the linear drive of the operating parameters for visualizing the filling level. According to the preferred embodiment, the translation position can be coupled preferably directly to the filling level of the hollow body. For example, the receiving region may have a loading contour corresponding to the shape of the hollow body to be received. In particular, a conveying plunger of the hollow body, such as the syringe plunger, can be connected to a translationally movable part of the linear drive, i.e., the spindle. In this way, a position of the linear drive is defined relative to the loading contour and, thus, relative to the hollow body. This offers the advantage that the translation position is coupled directly to the filling level so that the filling level can be easily determined and displayed.

According to the preferred embodiment, the drive may have a position sensor for detecting the translation position of the drive.

According to a development of the preferred embodiment, the position sensor may be suitable for directly detecting the translation position of the drive. Preferably, the position sensor may be designed as a potentiometer coupled to the translation position of the drive.

According to another development of the preferred embodiment, the position sensor may be suitable for detecting a reference translation position of the drive in a predetermined reference position, and the drive may include a rotation angle sensor for detecting the rotation angle position of the drive. Hence, the translation position can be determined indirectly by detecting the reference translation position corresponding to a reference position of the spindle and the syringe plunger, respectively, and by detecting the change of rotation angle of the drive motor corresponding to a distance covered of/travelled by the spindle and the syringe plunger, respectively. For example, the drive motor can be designed as a step motor and the rotation angle sensor can be suitable for detecting travelled motor steps of the step motor for detecting the rotation angle position of the drive. According to one embodiment, the drive can include a rotary encoder, in particular an encoder disk, connected to the drive motor, the rotation angle sensor being suitable for detecting the rotary encoder for detecting the rotation angle position of the drive. Preferably, the control unit may be configured to calculate the translation position of the drive based on the reference translation position and the motor steps covered.

According to a preferred embodiment, a feed or a rotational speed of the drive can be the operating parameter for visualizing the filling level. According to the preferred embodiment, the feed or the rotational speed can be coupled preferably directly with a change of filling level and a conveying rate of the hollow body, respectively. According to the preferred embodiment, the display unit can be configured to dynamically display the change of filling level and, resp., the conveying rate. Thus, the user can easily monitor the data relevant to the user.

According to a preferred embodiment, the control unit can be configured to prompt a user, preferably after closing the cover and/or in response to a predetermined user input, such as pressing a start button, to input and/or select, i.e., to select from a predetermined selection, a type of hollow body of the received hollow body, in particular a syringe size and/or a syringe manufacturer.

The control unit may include a memory.

According to a preferred embodiment, in the memory the hollow body data of the drive particularly relevant to conveying the liquid can be stored in response to the type of hollow body such as a length of a plunger rod of the hollow body. According to the preferred embodiment, the control unit can be configured to control the drive based on the hollow body data. In particular, in the memory a correlation between a feed or a rotational speed of the drive and a conveying rate of the type of hollow body can be deposited. Preferably, a correlation between an absolute position, i.e., in a linear drive between a translation position, and a residual fill volume of the hollow body type, i.e., the filling level, can be defined in the memory.

According to a preferred embodiment, image data of the hollow body types can be stored in the memory. According to the preferred embodiment, the control unit can be configured to display the image of the input and/or selected hollow body type, in particular in a realistic representation of the hollow body type, with a visualized filling level on the display unit. A realistic representation is to be understood to the effect that a scale, the visualized filling level and the hollow body type correspond, specifically true to scale, to the received hollow body and the filling level thereof. This offers the advantage that the filling level is monitored in a particularly intuitively comprehensible manner so that potentially occurring errors can be detected at an early stage.

According to a preferred embodiment, in the memory critical filling levels of the hollow body types can be stored. Alternatively, the control unit may be configured to prompt a user, preferably in response to a predetermined user input, to input and/or select the critical filling level of the hollow body. According to the preferred embodiment, the control unit can be configured to output, when the level falls below the critical filling level of a received hollow body, a preferably optical or acoustic signal, wherein the optical signal is preferably output on the display unit, such as in the form of a change of color of the visualized filling level, in the form of a flashing display of the visualized filling level or in the form of a warning message.

According to a preferred embodiment, the control unit can be configured to display the filling level of the hollow body on the display unit permanently or in response to a predetermined user input and/or a specific operating parameter of the drive or a specific state of the hollow body, e.g., when the level falls below the critical level. For example, the user can choose between different outputs on the display unit so that always the data relevant to the user can be displayed to the her/him. When, on the one hand, the filling level and other information are alternately displayed, the display unit can be minimized/can be kept small and the information can be displayed as large as possible on the existing display area.

According to a preferred embodiment, the control unit can be configured to prompt a user, preferably after closing the cover and/or in response to a predetermined user input such as pressing a start button, to input and/or select, i.e., select from a predetermined selection, which liquid is received in the hollow body.

According to a preferred embodiment, the information which liquids are critical can be deposited in the memory. According to the preferred embodiment, the control unit can be configured to output a preferably optical or acoustic signal when the liquid received in the hollow body is critical. Preferably, the optical signal can be output on the display unit, for example in the form of a change of color of the visualized filling level, in the form of a flashing display of the visualized filling level or in the form of a warning message.

According to a preferred embodiment, color codes of liquids may be stored in the memory. According to the preferred embodiment, the control unit can be configured to display a liquid designation or a background of the liquid designation and/or the filling level corresponding to the color code of the liquid contained in the hollow body on the display unit. In this way, a user can detect easily and intuitively which substance is currently being administered by the device.

The present disclosure also relates a method for operating a device for administering a medical liquid. The method comprises the following steps of:
receiving a liquid-containing hollow body, in particular a syringe, in the device,
operating a drive of the device to convey a liquid contained in the hollow body, in particular to move a plunger of the syringe,
detecting an operating parameter of the drive, in particular a translation position of the drive designed as linear drive, such as by directly detecting the translation position or by indirectly detecting a reference translation position and a relative change of position or change of rotation angle, respectively,
visualizing the filling level of the hollow body on the display unit based on the operating parameter of the drive.

In other words, the present disclosure relates to a device and a method for administering a medical liquid, in particular a syringe pump. Accordingly, the syringe is represented instead of the direct visibility in the syringe pump on the display unit (pump display). The syringe plunger is displayed dynamically according to the current filling level of the syringe. The filling level is determined via the position detection of the drive (pump drive, in particular linear drive) and is coupled to the syringe display. In this way, the user can view the filling level both directly at the syringe by opening the cover (operating front) to view the syringe lying there behind, and (indirectly) by the visualization on the display unit (in particular in the operating front).

Thus, the user can see the filling level of the syringe and the type of syringe without opening the door, which increases ergonomics, as the operating step for opening the door can be dropped/omitted. In addition, all data relevant to conveying can be seen at any time via the display unit. It is another advantage that in the device (apparatus design) no recess for viewing the syringe is required which would reduce the size of the display unit. Moreover, the filling level of the syringe can be detected even from a larger distance, such as from the end of a patient bed or in low light conditions, on the (illuminated) display unit.

More concretely, loading a syringe requires a selection of syringes on the display unit (which at the same time serves as a user interface/input unit) to determine the syringe size and the manufacturer of the syringe. The specific data relevant to the syringe are deposited in the memory of the device and have an influence on correct liquid conveying. The liquid conveying can be realized via a linear drive acting on the syringe so that the conveying rate results from a defined rotational frequency of the drive motor contained in the linear drive (electric motor) and a spindle translating the rotation to a translation. For example, step motors can be used. For correctly carrying out the functions of the device, the control unit must detect both the relative variation of the drive position, as the latter defines the conveying rate, and the absolute position, as the latter defines the still remaining liquid volume of the syringe. For detecting the drive position, detection via a position sensor is possible the signal of which directly indicates the drive position, for example using a potentiometer coupled to the drive position. Alternatively, it is possible to determine the drive position via one (or more) position sensor(s) in a defined position, e.g., as an electromechanical switch as end stop, in combination with the motor speed detection. For example, the steps of the step motor can be counted and/or an encoder disk connected to the drive motor can be evaluated. For this purpose, the control unit determines the absolute position of the drive by starting up the position sensor in a reference run and by a counter connected to the motor speed detection.

Moreover, the syringe can be loaded with its main part into a counter contour/loading contour of the device, and the syringe plunger can be connected to the movable part of the linear drive. In this way, the device and the control unit, respectively, can determine the position of the syringe plunger in the syringe main part and, thus, the filling level of the syringe, as the position of the linear drive is defined relative to the loading contour, the absolute position of the drive is known and geometric data of the selected syringe are deposited in the memory of the control unit. The device can visualize, based on the existing data, the filling level of the syringe on the display unit, wherein the visualized filling level preferably adjusts according to the dynamically conveyed amount of liquid and, resp., the variation of the drive position. The representation of the syringes adjusts corresponding to the selected type of syringe so that the user can detect the necessary features specific to the syringe type, such as the type designation and the scale. Via the selection of an operating element on the user interface, the syringe visualization can be displayed permanently or upon user request. The displayed liquid color in the syringe representation can be used as a signal for the user to increase the safety and/or ergonomics, for example by emphasizing a critical drug or by the color varying in response to the filling level.

### BRIEF DESCRIPTION OF THE DRAWING FIGURES

FIG. 1 shows a representation of a device for administering a medical liquid with a visualized filling level at a first point in time;

FIG. 2 shows a representation of the device for administering a medical liquid with the visualized filling level at a second point in time;

FIG. 3 shows a representation of the device for administering a medical liquid with the visualized filling level at a third point in time; and

FIG. 4 shows a schematic representation of a method for operating the device.

### DETAILED DESCRIPTION

In the following, a preferred embodiment of the present disclosure will be described on the basis of the associated figures.

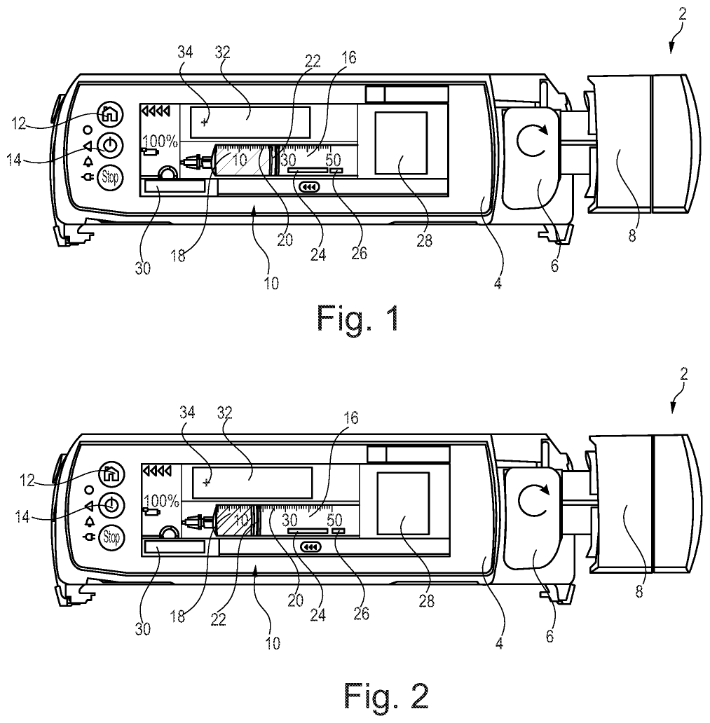

FIGS. 1 to 3 show a device 2 for administering a medical liquid according to the present disclosure. The device 2 is designed specifically as a syringe pump. The device 2 has a receiving region which is suitable for receiving a hollow body (not shown) containing the liquid to be administered. The device 2 includes a cover 4 which is connected to the receiving region movably between an opened position and a closed position. In the opened position of the cover 4, the receiving region is accessible for loading the hollow body, for example, and/or visible for reading a filling level of the liquid contained in the hollow body, for example. In the closed position of the cover 4, the receiving region, at least the filling level of the hollow body, is covered to the outside.

In FIGS. 1 to 3, the device 2 is shown in the closed position of the cover 4 so that the receiving region and the receivable or received hollow body are not shown, because they are hidden behind the cover 4. For opening and closing, the device 2 includes a lever 6 upon actuation of which, in particular by pulling on the same, the cover 4 is moved to the opened position and the hollow body can be loaded into the receiving region or can be removed from the receiving region. Also, the filling level of the hollow body can be read, in the opened position of the cover 4, directly on the hollow body.

The device 2 includes a drive 8. The drive 8 is suitable for acting on the hollow body received in the receiving region in such a way that the liquid contained in the hollow body is conveyed. The drive 8 may preferably be designed as a linear drive. For example, the linear drive can include a drive motor in the form of an electric motor and a spindle, wherein the spindle translates the rotational movement of the drive motor into a translation movement. The translation movement is coupled preferably directly with the conveying of the liquid contained in the hollow body.

The device 2 includes a display unit 10 for graphically displaying the filling level of the hollow body. In particular, the display unit 10 is designed as a display. Preferably, the display unit 10 can also be used as an input unit and can be designed as a touch display, for example. The display unit 10 in the shown embodiment is integrated in the cover 4 on an outside of the device 2. Thus, the display unit 10 is oriented, in the position of use of the device 2, to the front toward the user and is clearly visible. As an alternative, it is also possible to dispose the display unit 10 in an external display device.

The display unit 10 is suitable for displaying the filling level of the hollow body on the basis of one or more operating parameter(s) of the drive 8 particularly relevant to conveying the liquid. In particular, a translation position of the drive 8 designed as linear drive can be an operating parameter for visualizing the filling level. The translation position of the linear drive is preferably directly coupled to the filling level of the hollow body, for example by a plunger disposed axially movably inside the hollow body for conveying the liquid being connected to the spindle of the drive 8.

The drive 8 may have a position sensor for detecting the translation position of the drive. The position sensor may be suitable for directly detecting the translation position of the drive. Alternatively, the position sensor can be suitable for indirectly detecting the translation position of the drive by detecting a reference translation position of the drive in a predetermined reference position, and the drive including a rotation angle sensor for detecting the rotation angle position of the drive on the basis of which the (absolute) translation position of the drive and thus the filling level can be determined.

In particular, a feed and/or a rotational speed of the drive 8 can be an operating parameter for visualizing the filling level. Accordingly, the feed and/or the rotational speed is/are coupled preferably directly with a change of the filling level and a (liquid) conveying rate of the hollow body, respectively. For example, the display unit can be configured to dynamically display the change of filling level and the conveying rate, respectively.

According to a preferred embodiment, the device 2 can include a control unit for controlling the drive. Preferably, the control unit can be configured to prompt a user to input and/or select a hollow body type of the received hollow body, in particular a size, a volume and/or a manufacturer. For example, the control unit can prompt the user to carry out an input after closing the cover and/or in response to a predetermined user input such as pressing a start button/field/start key 12 or an on-off-button/field/on-off-key 14.

According to a preferred embodiment, the control unit can include a memory. In the memory, hollow body data depending on the type of hollow body can be stored. For example, hollow body data relevant to conveying the liquid can be stored, such as a length of a hollow body plunger rod. Preferably, the control unit can be configured to control the drive based on the hollow body data. In particular, a correlation between a feed or a rotational speed of the drive and a change of filling level/conveying rate of the type of hollow body can be deposited in the memory. Preferably, a correlation between an absolute position, i.e., for a linear drive between a translation position, and a residual fill volume of the hollow body type, i.e., the filling level, can be defined in the memory.

According to a preferred embodiment, image data of the hollow body types can be stored in the memory. Preferably, the control unit can be configured to display on the display unit 10 the image of the input and/or selected hollow body type, in particular in a realistic representation of the hollow body type, with a visualized filling level and with the same orientation/alignment as the hollow body/syringe located behind the cover 4. This is obvious in the embodiment shown in FIGS. 1 to 3. On the display unit 10, a visualized syringe 16 is displayed which corresponds realistically to the selected type of hollow body. A visualized filling level 18 is shown inside the syringe 16. For this purpose, a scale 20 of the fill volume as well as a visualized syringe plunger position 22 is indicated on the syringe 16. On the display unit 10, the syringe 16 is designated with its type designation 24 and its maximum volumes 26 which correspond to the hollow body type input and, resp., to the hollow body data deposited in the memory.

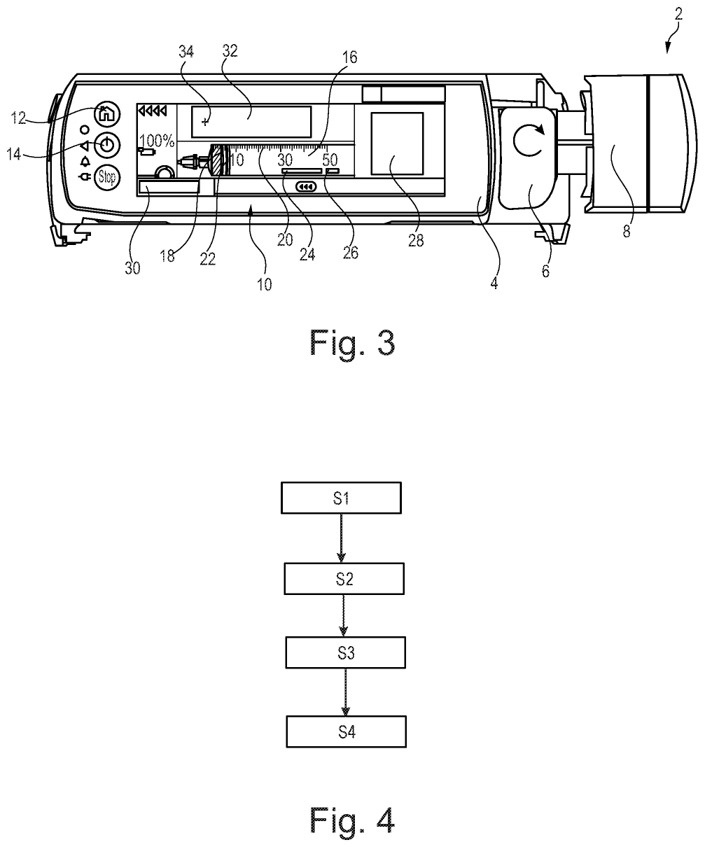

Depending on the operating parameters of the drive 8, the filling level 18 is dynamically visualized. In FIGS. 1 to 3, the filling level 18 is shown at different points in time, wherein the filling level 18 decreases between the representation in FIG. 1 and FIG. 3. In addition, a visualized conveying rate 28 is shown, e.g., in ml/h, on the display unit 10.

According to a preferred embodiment, critical filling levels of the types of hollow body can be stored in the memory. The control unit can preferably be configured to output, when the level falls below the critical filling level, e.g., 5 ml, of the hollow body, a preferably optical or acoustic signal. For example, the optical signal can be output on the display unit 10 for example in the form of a change of color of the visualized filling level 18, in the form of a flashing display of the visualized filling level 18 or in the form of a warning message.

According to a preferred embodiment, the control unit can be configured to permanently display the visualized filling level 18 of the hollow body on the display unit 10. Alternatively, the control unit can be configured to display the visualized filling level 18 of the hollow body on the display unit 10 in response to a predetermined user input, such as when the display change button 30 is actuated, and/or when the level falls below the critical filling level. For example, the user can choose between different outputs on the display unit 10 via the display change button 30.

According to a preferred embodiment, the control unit can be configured to prompt the user to input and/or select, i.e., to select from a predetermined selection, which liquid is received in the hollow body. The control unit is preferably configured to visualize the selected liquid on the display unit 10 by displaying a liquid designation 32. For example, the control unit can be configured to prompt the user to select the appropriate liquid for inputting the liquid in response to a predetermined user input, for example when actuating a liquid select key/a liquid select field 34.

In particular, color codes of liquids can be stored in the memory. According to a preferred embodiment, the control unit can be configured to display on the display unit 10 the liquid designation 32 or a background of the liquid designation 32 and/or the filling level 18 corresponding to the color code of the liquid contained in the hollow body.

Of preference, characteristics of the liquids can be deposited in the memory, such as which liquids are critical. Preferably, the control unit can be configured to output a preferably optical or acoustic signal, if the liquid received in the hollow body is critical. The optical signal can preferably be output on the display unit for example in the form of a change of color of the visualized filling level 18, in the form of a flashing display of the visualized filling level 18 or in the form of a warning message.

The present disclosure also relates to a method for operating the device 2 which is shown in 5 FIG. 4. In a first step S1 of the method, a liquid-containing hollow body is received in the device. In a second step S2, the drive 8 of the device 10 is operated to convey a liquid contained in the hollow body. In a third step S3, one or more operating parameter(s) of the drive 8 is/are detected. In particular, a translation position of the drive 8 designed as linear drive and/or a feed or a rotational speed of the drive 8 is/are detected. In a fourth step S4, the filling level of the hollow body is 10 displayed on the display unit 10 based on the one or more operating parameter(s) as the visualized filling level 18.

1. A device for administering a medical liquid, n the device comprising:
a receiving region for receiving a hollow body containing the medical liquid;
a cover connected to the receiving region and movable between an opened position and a closed position;
a drive configured for acting on the hollow body when the hollow body is received in the receiving region such that the medical liquid is conveyed; and
a display unit for graphically displaying a filling level of the hollow body.
the display unit configured for displaying the filling level of the hollow body based on one or more operating parameters of the drive,
the receiving region being accessible when the cover is in the opened position, and
at least the filling level of the hollow body being covered by the cover when the cover is in the closed position.

2. The device according to claim 1, wherein the drive is designed as a linear drive, and
wherein the one or more operating parameters of the drive comprises a translation position of the linear drive,
the translation position of the linear drive being a function of the filling level of the hollow body.

3. The device according to claim 2,
wherein the drive includes a position sensor for detecting the translation position of the drive, the position sensor being configured for:
a) directly detecting the translation position of the drive, or
b) detecting a reference translation position of the drive in a predetermined reference position, and
wherein the drive includes a rotation angle sensor for detecting a rotation angle position of the drive.

4. The device according to claim 1, wherein the one or more operating parameters of the drive comprises a feed and/or a rotational speed of the drive, the feed and/or the rotational speed being a function of a change of filling level of the hollow body.

5. The device according to claim 1, further comprising a control unit configured to prompt a user to input and/or select a specific hollow body type corresponding to the hollow body.

6. The device according to claim 5,
wherein the control unit includes a memory in which hollow body data specifically relevant to conveying the medical liquid are stored, and
wherein the control unit is configured to control the drive based on the hollow body data.

7. The device according to claim 6,
wherein image data of hollow body types are stored in the memory, and
wherein the control unit is configured to display an image of the specific hollow body type on the display unit, with a visualized filling level.

8. The device according to claim 7,
wherein critical filling levels of the hollow body types are stored in the memory,
wherein the critical filling levels include a critical filling level of the specific hollow body type, and
wherein the control unit is configured to output a signal when the filling level of the hollow body falls below the critical filling level.

9. The device according to claim 8, wherein the control unit is configured to display the filling level of the hollow body on the display unit permanently or in response to a predetermined user input and/or when the filling level of the hollow body falls below the critical filling level.

10. The device according to claim 6,
wherein color codes of medical liquids are stored in the memory, and
wherein the control unit is configured to display a liquid designation or a background of the liquid designation and/or a filling level corresponding to the color code of the medical liquid contained in the hollow body.

11. A method for operating a device for administering a medical liquid the method comprising the steps of:
receiving a hollow body in the device, the hollow body containing the medical liquid;
operating a drive of the device to convey the medical liquid;
detecting one or more operating parameters of the drive; and
visualizing a filling level of the hollow body on a display unit based on the one or more operating parameters of the drive.

12. The device according to claim 1, wherein the receiving region is accessible in the opened position of the cover for loading the hollow body and/or is visible for reading the filling level of the medical liquid.

13. The device according to claim 1, wherein the one or more operating parameters of the drive are relevant to conveying the liquid.

14. The device according to claim 2, wherein the translation position of the linear drive is directly related to the filling level of the hollow body.

15. The device according to claim 4, wherein the filling level, the feed and/or the rotational speed is/are directly related to the change of filling level of the hollow body.

16. The device according to claim 5, wherein the control unit is configured to prompt the user to input and/or select the specific hollow body type corresponding to the hollow body after the cover is moved to the closed position and/or in response to a predetermined user input.

17. The device according to claim 5, wherein the specific hollow body type comprises one or more of a size, a volume and/or a manufacturer of the hollow body.

18. The device according to claim 8, wherein the signal is an optical or an acoustic signal.

19. The device according to claim 1, wherein the device is a syringe pump.

20. The method according to claim 11, wherein the step of detecting one or more operating parameters of the drive comprises detecting a translation position of the drive.
